(19) United States
(12) Patent Application Publication
(10) Pub. No.: US 2008/0059225 A1
(43) Pub. Date: Mar. 6, 2008

(54) Health management cyclic service method of a community association and system of using the same

(76) Inventor: Fong-Chyi Chang, Taichung City (TW)

Correspondence Address: COOPER & DUNHAM, LLP, 1185 AVENUE OF THE AMERICAS, NEW YORK, NY, 10036, US

(21) Appl. No.: 11/515,490

(22) Filed: Sep. 1, 2006

(51) Int. Cl. G06Q 10/00; G09B 23/28; A61B 5/00

(52) U.S. Cl. 705/2; 434/262; 600/300

(57) ABSTRACT

A health management cycling service method and system for a community association has five steps to scheme a personal health management plan according to each inhabitant's conditions of the life history, living environment, and the situations of body and mind. In addition, the business actively monitors and records the progresses of executing his or her personal health management plan to obtain a better management effect. The inhabitant can repeats the five steps to progressively and effectively reduce the number and the level of any abnormal health problem and finally reaching the best healthy situation.

### BACKGROUND OF THE INVENTION

1. Field of the Invention

The present invention relates to a health management cycling service method and system of a community association, and more particularly to a cycling service method and system of managing inhabitant health for a community association.

2. Description of Related Art

In present, most people would like to pay more attention on his or her body and mind health and some health management companies or organizations provide personal health management to monitor people's body and mind health. However, the health management services are too expensive and the people who live in busy city have no time to understand the health management, so the health management service is not general. Therefore, most people go to hospital when some abnormal symptoms of their body or mind are happened.

Further, since the health management provider schemes the personal health management based on the customer's present body and mind situation and does not further consider other community and work environment factors, the management effect is not good enough. In addition, the provider does not provide the customer an immediate, necessary and correct information about health and hygiene, a professional consulting service with doctor or consultant and a domestic medical care service. Furthermore, the traffic between home and the health management company will spend a lot of money and time.

Therefore, the present invention establishes a health management cyclic service system for community association to provide the community inhabitant exactly manage and understand their body condition. Further, the present invention provides a cyclic service method of using the health management cyclic service system to actively provide the community inhabitant a convenient and efficient health management service, and have cost down for buying a healthcare food or medication etc. medical product.

### SUMMARY OF THE INVENTION

The main objective of the present invention is to provide a health management cyclic service method and system for a community association.

The health management cyclic service method has five steps to scheme a personal health management plan according to each inhabitant's conditions of the life history, living environment, and body and mind. In addition, the business actively monitors and records the progresses of executing his or her personal health management plan to obtain a better management effect. The inhabitant can repeat the five steps to progressively and effectively reduce the number of abnormal items of cyclic health examining results.

The health management cyclic service system for a community association has a center server building a health management method, a professional consulting server linking to the center server, and an internet bank trading server liking to the center server. The center sever provides community inhabitants a personal health management service through the community management server. The community inhabitants use computer to link to these servers can obtain personal health examination list, a health management service and knowledge about medical. Therefore, the community inhabitants obtain better and convenient health management and save a lot of time to consult with a doctor or order a health examination procedure.

Other objectives, advantages and novel features of the invention will become more apparent from the following detailed description when taken in conjunction with the accompanying drawings.

### BRIEF DESCRIPTION OF THE DRAWINGS

FIG. 1 is a block diagram of the health management cyclic service system in accordance with the present invention;

FIGS. 2A and 2B are a detailed block diagram of a health management center server of the health management system in accordance with the present invention;

FIGS. 3A and 3B are a detailed block diagram of one community management server connected to multiple devices in a community;

FIG. 4 is a block diagram of a common configuration for multiple health management equipments and the community management server in the community in accordance with the present invention;

FIG. 5 is a block diagram of a host controller of each health management equipment in accordance with the present invention;

FIG. 6 is a flow chart of a health management procedure build in a center server of the health management cyclic service system in accordance with the present invention.

FIG. 7 is a flow chart of a health management cyclic service method in accordance with the present invention;

FIG. 8 is a detailed functional block diagram of the health management cyclic service method in accordance with the present invention;

FIGS. 9A to 9E are detailed flow charts of the health management cyclic service method in accordance with the present invention; and

FIG. 10 is a list of a knowledge suggestion list generated by the health management cyclic service method in accordance with the present invention.

### DETAILED DESCRIPTION OF THE PREFERRED EMBODIMENT

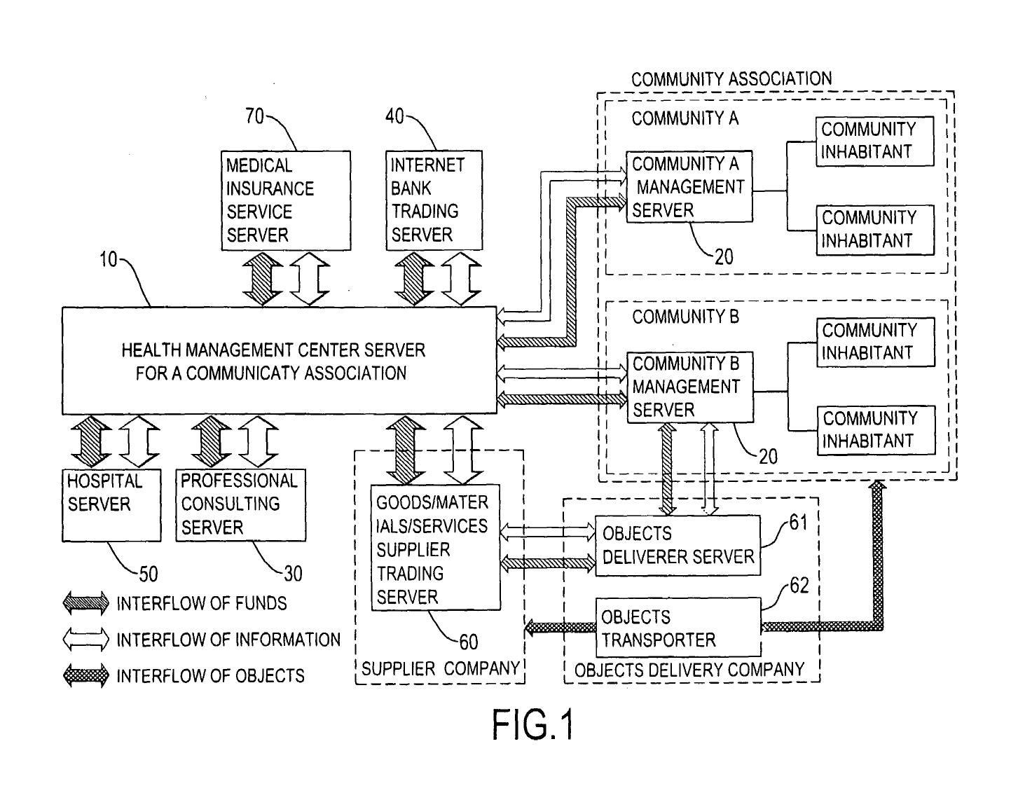

With reference to FIG. 1, a block diagram of a health management cyclic service system in accordance with the present invention has a health management center server (10) for a community association, a professional consulting server (30), an internet bank trading server (40), a hospital server (50), a goods/materials/services supplier web trading server (60) in a supplier company and a medical insurance service server (70). The center server (10) builds a health management procedure and is linked to the professional consulting server (30), the internet bank trading server (40), the hospital server (50), the goods/materials/services supplier web trading server (60) and the medical insurance service server (70). The center server (10) is used to link to at least one community management server (20) through Internet. Therefore, the community inhabitant uses the community management server (20) to like to the center server (10). The goods/materials/services supplier web trading server (60) provides further links to an objects deliverer server (61) in a objects delivery company to notify a objects transporter (62) of the objects delivery company to delivery the goods, materials and services ordered by the inhabitant or the health management center server (10).

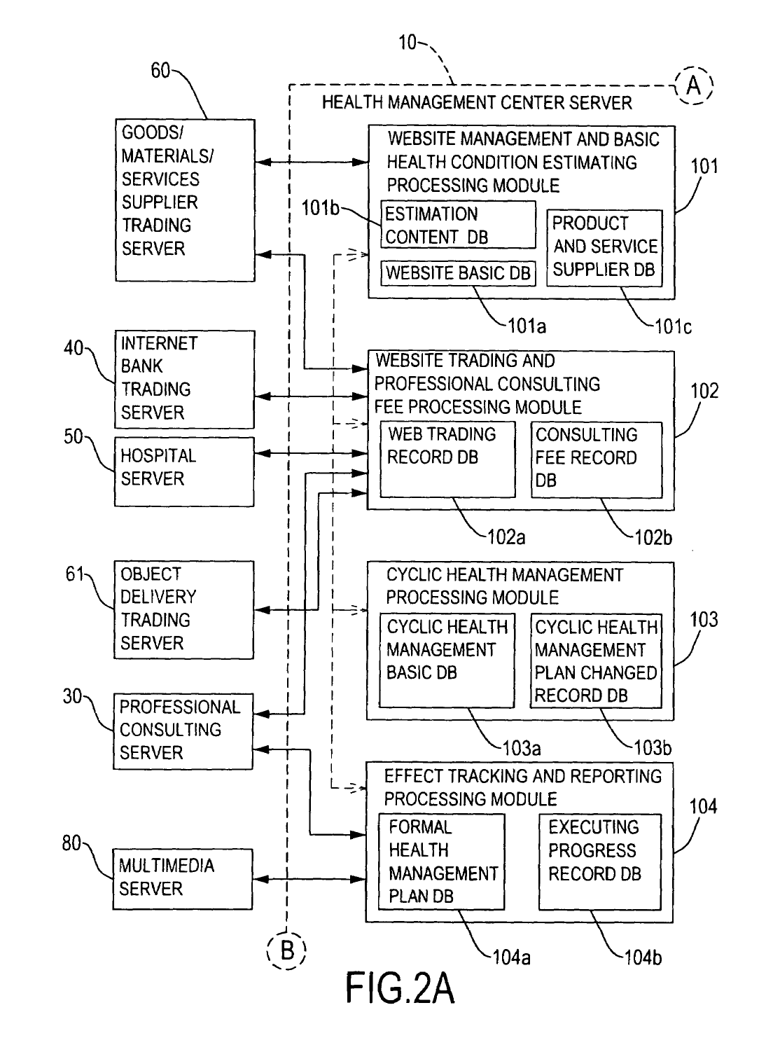

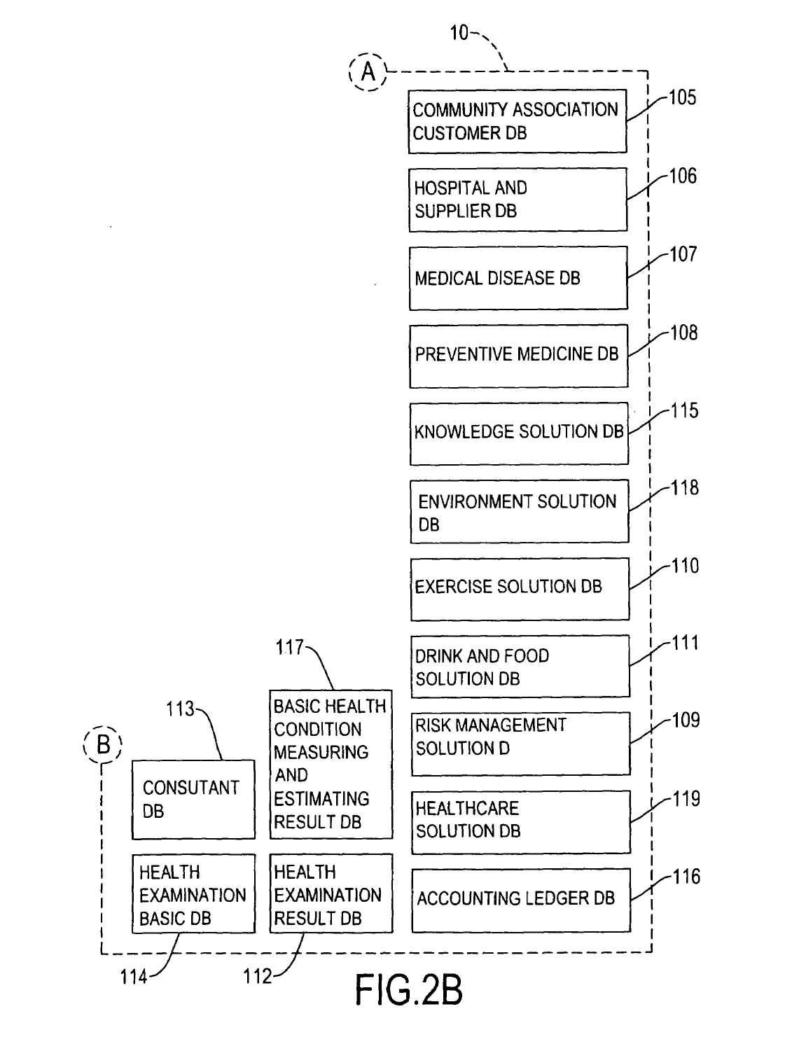

With further reference to FIGS. 2A and 2B, the center server (10) further builds a community association customer database (105), a hospital and supplier database (106), a medical disease database (107), a preventive medicine database (108), a risk management solution database (109), an exercise solution database (110), a drink and food solution database (111), a result of health examination database (112), a consultant database (113), a health examination basic database (114), a knowledge solution database (115), an accounting ledger database (116), a basic health condition measuring/estimating result database (117), an environment solution database (118) and a healthcare solution database(119). Further, the center server (10) has a website management and basic health condition estimating processing module (101), a website trading and professional consulting fee processing module (102), a cyclic health management processing module (103) and an effect tracking and reporting processing module (104).

The website management and basic health condition estimating processing module (101) has a website basic database (101a), an estimation content database (101b) and a product and service supplier database (101c). The website trading and professional consulting fee processing module (102) has a web trading record database (102a) and a consulting fee record database (102b). The cyclic health management processing module (103) has a cyclic management basic database (103a) and a cyclic health management plan changed record database (103b). The effect tracking and reporting processing module (104) has a formal health management plan database (104a) and an executing progress record database (104b).

The website management and basic health condition estimating processing module (101) is used to link to the professional consulting server (30). The website trading and professional consulting fee processing module (102) is used to link to the internet bank trading server (40), a goods/materials/services supplier web trading server (60) and a multimedia server for health and hygiene education (80). The cyclic health management processing module (103) is used to link to the hospital server (50).

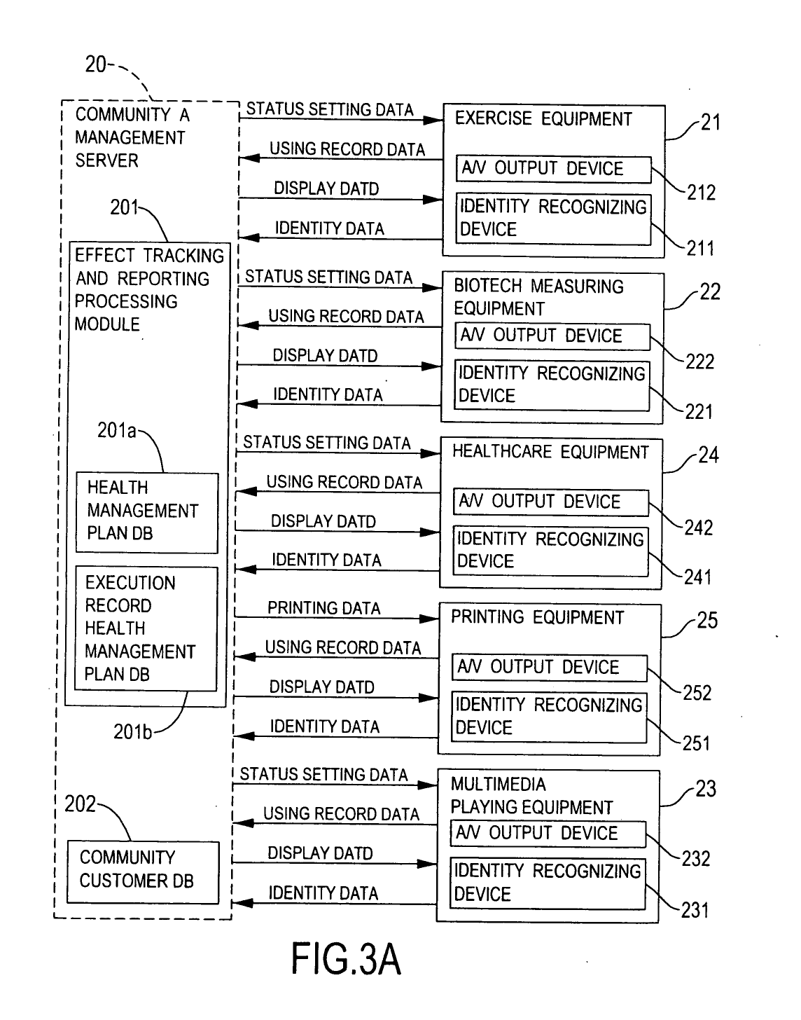

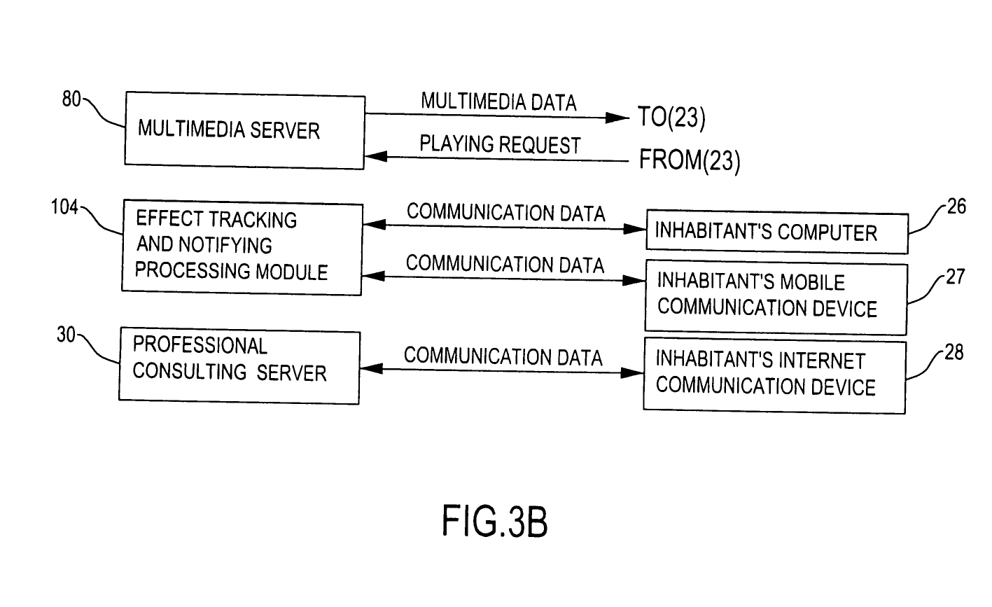

With reference to FIGS. 3A and 3B, each community management server (20) has an effect tracking and reporting processing module (201) and a community customer database (202). The effect tracking and reporting processing module (201) has a health management plan database (201a) and an executing record of health management plan database (201b).

The community management server (20) is used to link to exercise equipment (21), biotech measuring equipment (22), multimedia playing equipment (23), healthcare equipment (24), and printing equipment (25). The multimedia playing equipment (23) is further connected to a multimedia server (80) storing health and hygiene education data. The center server (10) controls the multimedia server (80) to display the health and hygiene education data according to the community inhabitant's identity. Therefore, the community inhabitant can gradually study knowledge about the health and hygiene when he or she uses the exercise equipment (21) or biotech measuring equipment (22). Further, the community inhabitant uses his or her computer (26) and mobile phone (27) to link to the effect tracking and reporting processing module (104) of the center server (10) and uses VoIP phone (28) to link to the professional consulting server (30).

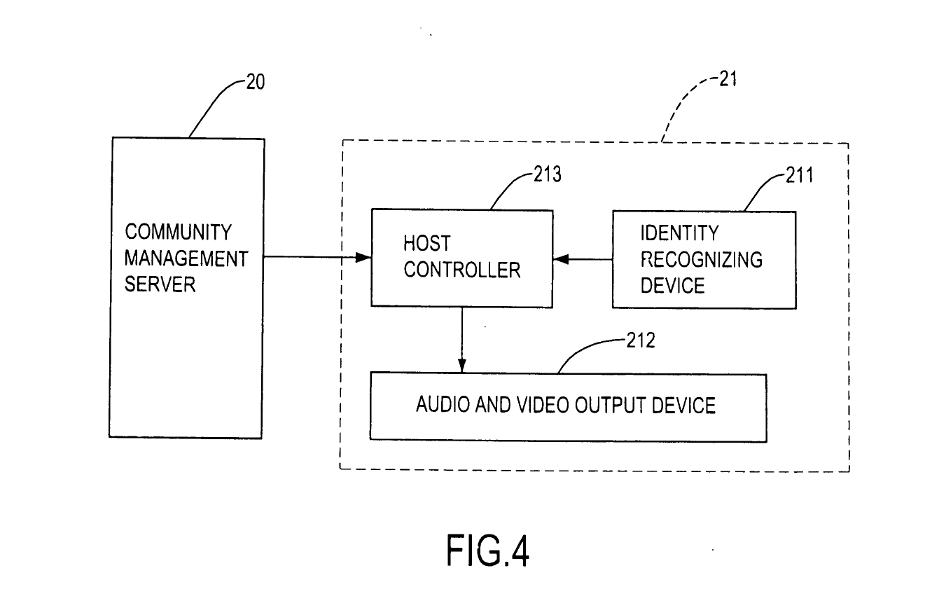

All health management equipment (21, 22, 23, 24, 25) have a common configuration. With further reference to FIG. 4, only shows the configuration of the exercise equipment (21) having a host controller (213), an identity recognizing device (211), an audio and video output device (212) and a specific health management function module (214). Before the community inhabitant uses the exercise equipment (21), the community inhabitant firstly uses the identity recognizing device (211) to verify his or her identity. When the community inhabitant uses the identity recognizing device (211), the identity recognizing device (211) provides the center server (10) the community inhabitant's identity through the community management server (20). Since the center server (10) builds the community association customer database (105), the center server (10) will check whether the community inhabitant had been registered. Therefore, the center server (10) can monitor and manage each community inhabitant's use conditions and executed records.

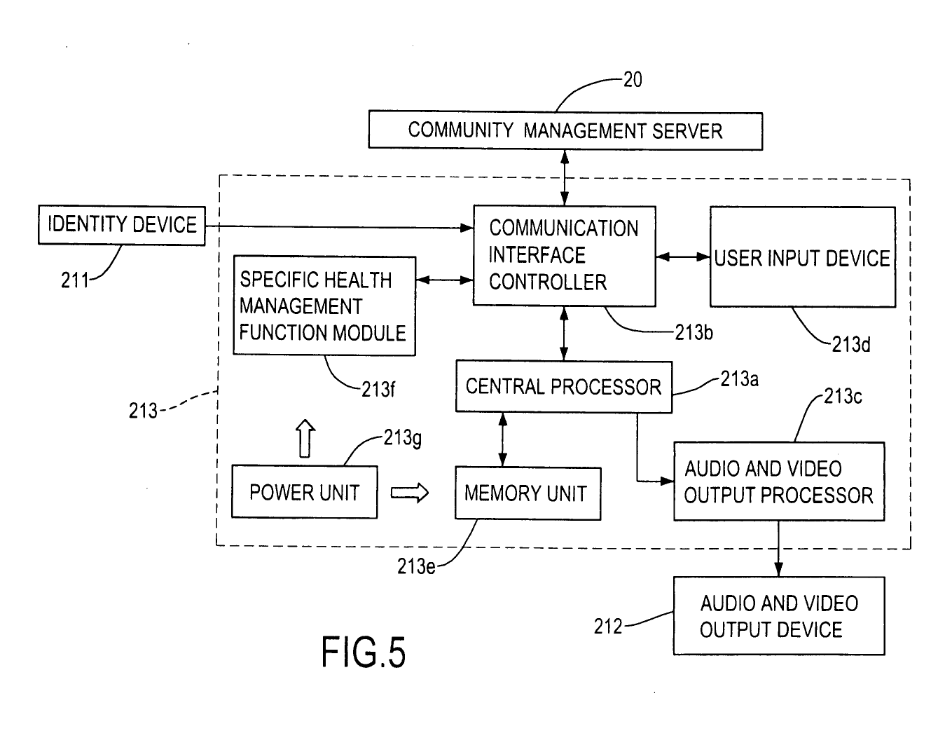

With reference to FIG. 5, the host controller (213) has a central processor (213a) with a memory unit (213e), a communication interface controller (213b), an audio and video output processor (213c), a user input device (213d), a specific health management function module (213f) and a power unit (213g). The central processor (213a) links to the communication interface controller (213b) and the audio and video output processor (213c). The audio and video output processor (213c) outputs audio/video signals to the audio and video output device (212). The identity recognizing device (211), the user input device (213d), and the specific health management function module (213f) link to the communication interface controller (213b), and the communication interface controller (213b)links to the community management server (20), so the community management server (20) would obtains the user's identity, use conditions and executed records of each health management equipment (21, 22, 23, 24, 25).

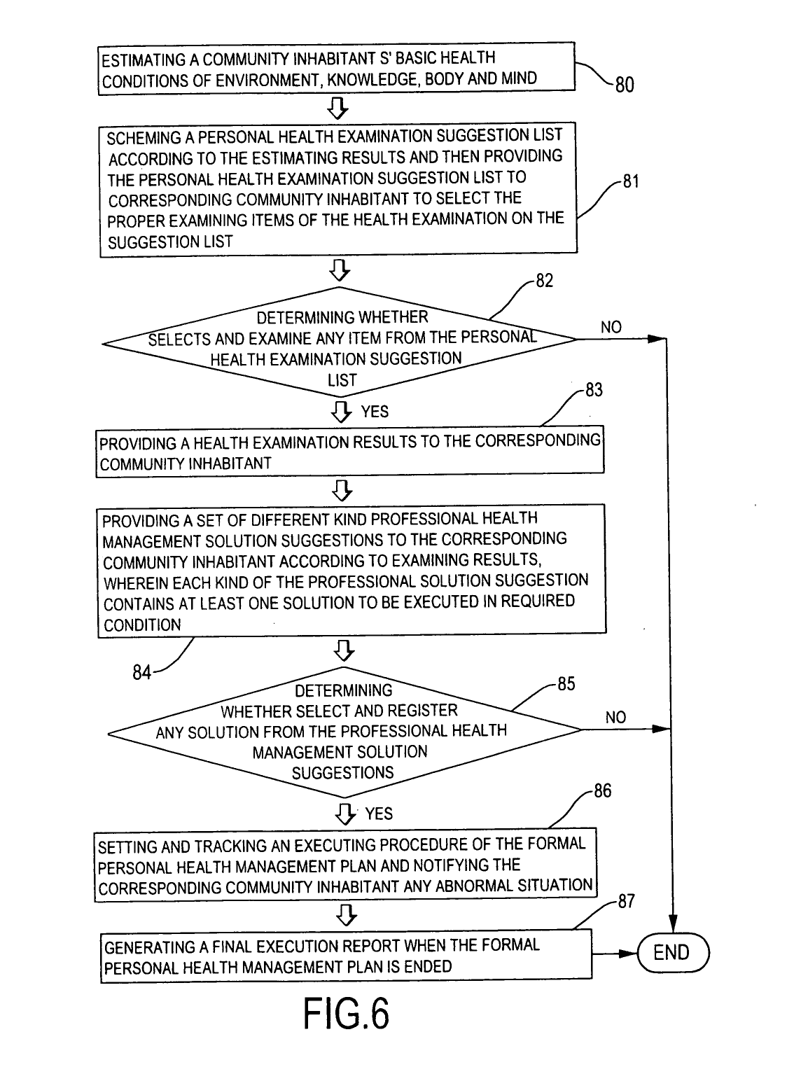

With reference to FIG. 6, the health management procedure of the center server has steps of:

(a) estimating a community inhabitants' basic health conditions of environment, knowledge, body and mind (80);

(b) scheming a personal health examination suggestion list according to the estimating results and then providing the personal health examination suggestion list to corresponding community inhabitant to select the proper examining items of the health examination on the suggestion list (81);

(c) determining whether the community inhabitant selects and examines any examining item from the personal health examination suggestion list, wherein if the community inhabitant does not select any examining item and then the health management procedure is end, but if the community inhabitant selects at least one examining item on the health examination suggestion list, the next step is executed (82);

(d) providing a health examination results to the corresponding community inhabitant (83);

(e) providing a set of different kind professional health management solution suggestions to the corresponding community inhabitant according to examining results, wherein each kind of the professional solution suggestion contains at least one solution to be executed in required condition (84);

(f) determining whether the community inhabitant selects and registers any solution from the professional health management solution suggestions, wherein if the community inhabitant does not register any solution and then the health management procedure is end, but if the community inhabitant registers at least one solution, a formal personal health management plan is schemed according to the selected solutions and then the nest step is executed (85);

(g) setting and tracking an executing procedure of the formal personal health management plan and notifying the corresponding community inhabitant any abnormal situation (86); and

(h) generating a final execution report when the formal personal health management plan is end (87).

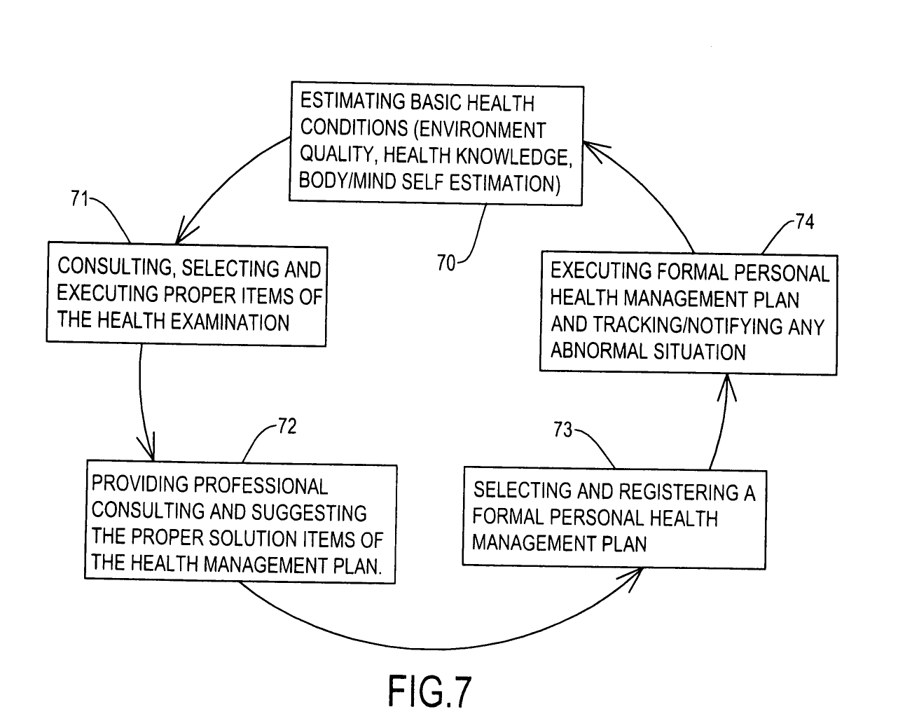

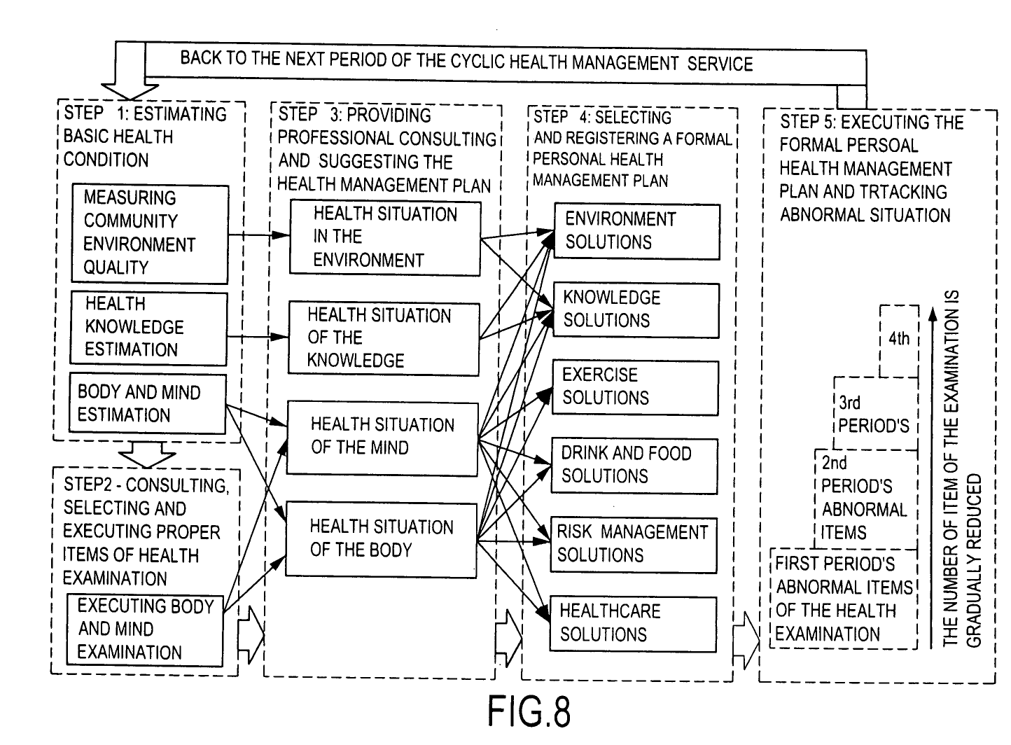

With reference to FIGS. 7 and 8, a flow chart of a health management cyclic service method in accordance with the present invention includes steps of:

(a) estimating basic health conditions of the inhabitant including community environment quality, health knowledge level and body and mind health (70);

(b) consulting and selecting proper items of a health examination to examine the actual inhabitant's body and mind (71);

(c) providing professional consulting and suggesting for the proper solution items of the health management plan according to the estimating results in steps (a) and (b), wherein the suggesting results includes environment suggestions, knowledge suggestions, exercise suggestions, drink and food suggestions, risk suggestions and healthcare suggestion (72);

(d) selecting and registering a formal personal health management plan according to the professional consulting and suggesting results (73); and

(e) executing formal personal health management plan and reporting any abnormal situation executing effects (74), wherein the executing formal personal health management plan including setting and tracking the executing procedure to find out the abnormal situation.

If the inhabitant circularly executes the above five steps, the number of the abnormal items to be examined is decreasing according to the good executing effects. That is, the present invention actively monitors and manages the inhabitants' health.

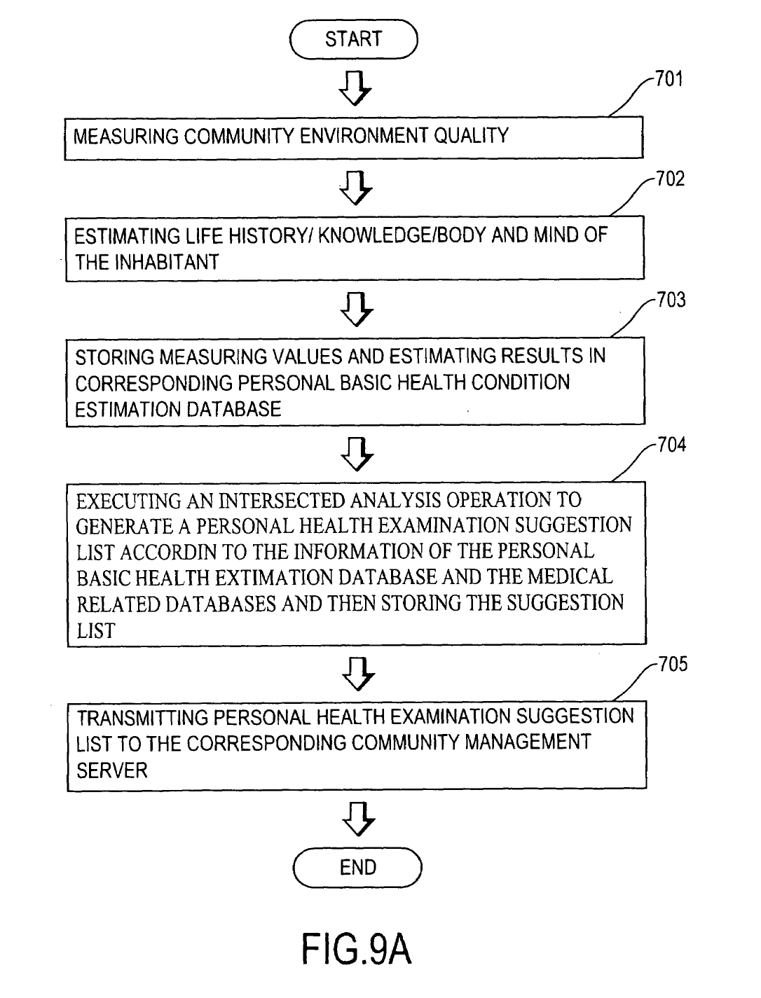

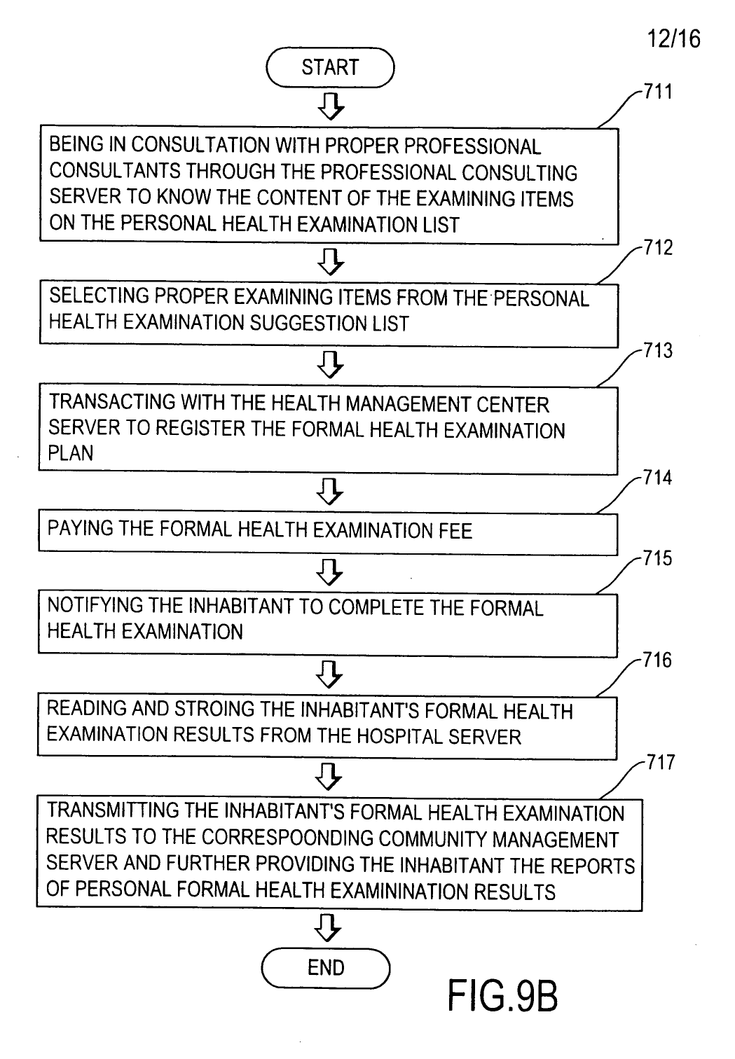

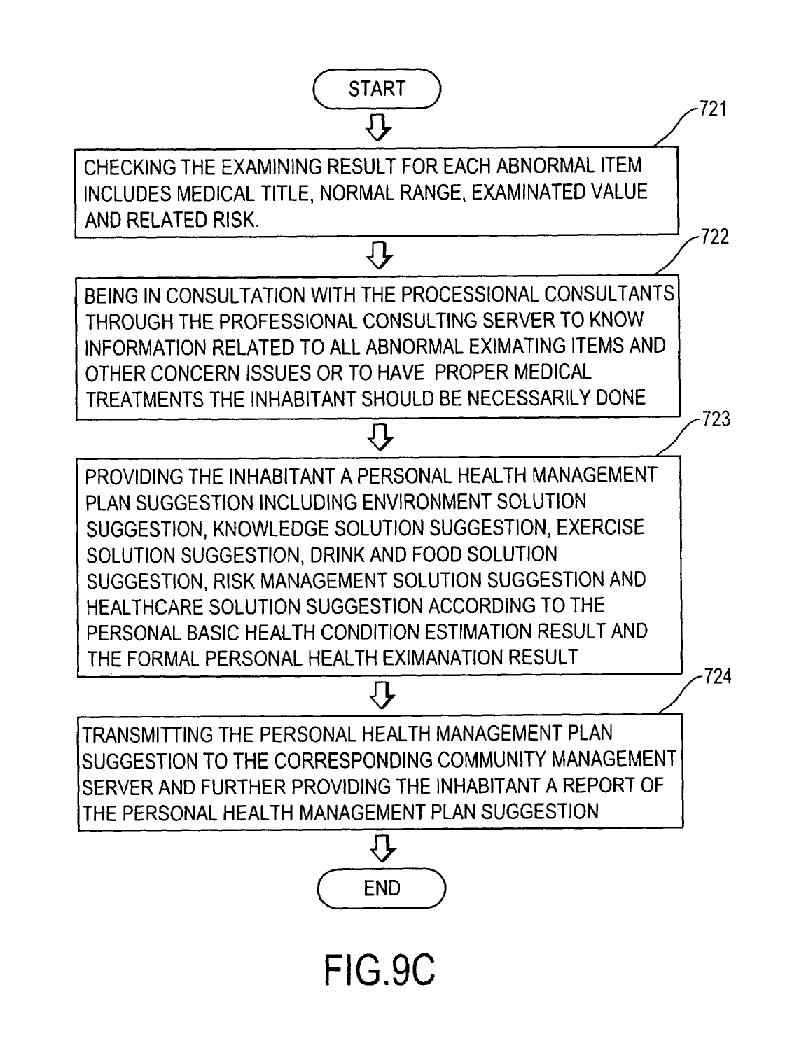

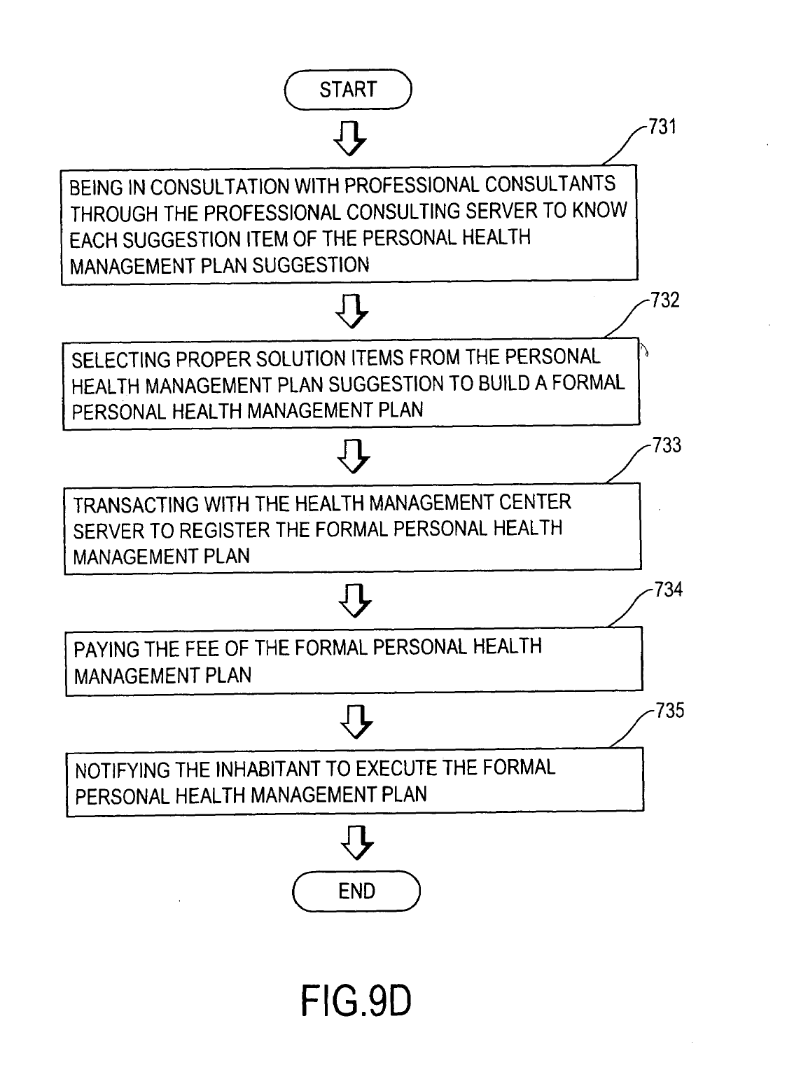

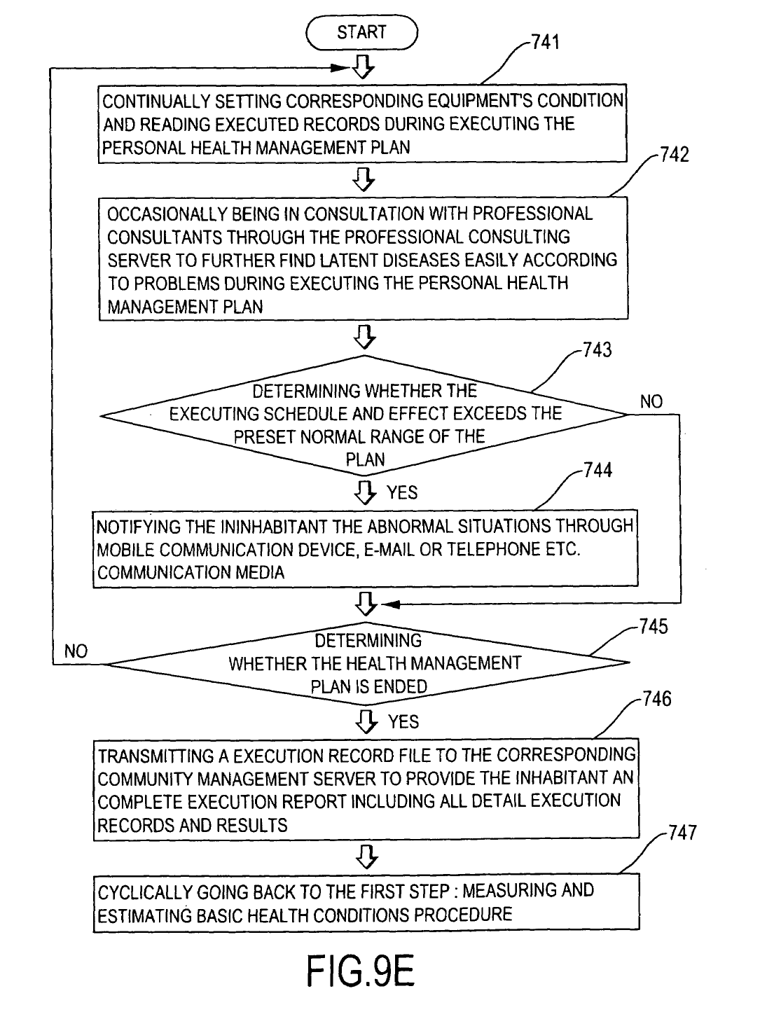

With reference to FIG. 9A, the step (a) has acts of:

(1) measuring community environment quality (701);

(2) estimating health conditions of life history, knowledge, and body and mind of the inhabitant (702);

(3) storing measuring values and estimating results in the personal basic health condition estimation database (703);

(4) executing an intersected analysis operation to generate a personal health examination suggestion list according to the information of the personal basic health condition estimation database (117) and the medical related databases and then storing the suggestion list, wherein the medical related databases has the medical disease database (107), the preventive medicine database (108), and then storing the personal health examination list (704);

(5) transmitting a personal health examination suggestion list to community management server which the inhabitant uses (705); and

(6) end the step (a).

The step (a) first estimates the inhabitant's related life and body and mind information, and then arranges the inhabitant to execute a personal health examination suggestion list based on the estimating results.

With further reference to FIG. 9B, the step (b) has acts of:

(a) being in consultation with a proper professional consultant through the professional consulting server to know the content of the related examining items on the personal health examination suggestion list (711);

(b) selecting proper examining items on the personal health examination suggestion list (712);

(c) transacting with the health management center server to register the formal health examination plan (713);

(d) paying the formal health examination fee (714);

(e) notifying the inhabitant to complete the formal health examination (715);

(f) reading and storing the inhabitant's formal health examination results from the hospital server (716);

(g) transmitting the inhabitant's formal health examination results to the corresponding community management server and further providing the inhabitant the reports of personal formal health examination results (717); and

(b) end the step (b).

The step (a) provides the inhabitant his or her personal health examination suggestion list, so the step (b) provides the inhabitant a website to order details of executing the personal health examination list. Therefore, the inhabitant not only understands his or her health but also orders quickly his or her own health examination items in the hospital through Internet. After the inhabitant completes the examination items in the hospital, the hospital server (50) stores the examining results. The inhabitant will get his or her report of the formal health examination results through the corresponding community management server.

With further reference to FIG. 9C, the step (c) has acts of:

(1) checking the examining result for each abnormal item, wherein the examining result shows medical title, normal range, examined value and related risk (721);

(2) being in consultation with consultants through the professional consulting server to know information related to all abnormal examining items or to have proper medical treatments, the inhabitant should be necessarily done (722);

(3) providing the inhabitant a personal health management plan suggestion including environment solution suggestion, knowledge solution suggestion, exercise solution suggestion, drink and food solution suggestion, risk solution suggestion and health care solution suggestion according to the personal basic health condition estimation result and the formal personal health examination result (723);

(4) transmitting the personal health management plan suggestion to the corresponding community management server and further providing the inhabitant a report of the personal health management plan suggestion (724); and

(5) end the step (c).

After the inhabitant checks the examining results generated from the step (b), the inhabitant will find some results of the examining items has the abnormal detecting values or high lethal ratio. At the time, the inhabitant would like to understand what the examining items with bad results are. Therefore, the step (c) provides the inhabitant a convenient way to consult the related information of the examining items and then the consultant records the consulting content and suggestions given to the inhabitant. Therefore, the professional consulting server (30) stores the consulting content and suggestions. The center server (10) reads the consulting content and suggestions and transmits the personal health management plan suggestion to the community management server (20). The inhabitant uses the community management server (20) to print the personal health management plan suggestion and then knows what kinds of examining items should be made to keep body and mind in health.

With reference to FIG. 9D, the step (d) has acts of:

(1) being in consultation with professional consultants through the professional consulting server to know each suggestion item (731);

(2) selecting proper suggestion items of personal health management plan suggestion list to build a formal personal health management plan (732);

(3) transacting with the health management center server to register the formal personal health management plan (733);

(4) paying the fee of the formal personal health management plan (734);

(5) notifying the inhabitant to execute the formal personal health management plan (735); and

(6) end step (d).

The step (d) provides the inhabitant a convenient way to order suggestion items to scheme the personal health management plan. If the inhabitant hardly to decide which suggestion items should be done, he or she could link to the professional consulting server to do consulting procedure. When the inhabitant decides the suggestion items or other items, the inhabitant logs in and registers the website provided by the center server (10) to select suggestion items to complete his or her own personal health management plan. After paying the management service fee, the center server will start the personal health management plan with the exercise equipment, the biotech measuring equipment, the healthcare equipment and the multimedia playing equipment through the community management server (20).

With reference to FIG. 9E, the step (e) has acts of:

(1) continually setting corresponding equipment's condition and reading executed records during executing the personal health management plan (741);

(2) occasionally being in consultation with professional consultants through the professional consulting server to further fine latent diseases easily according to problems during executing the personal health management plan (742);

(3) determining whether the executing schedule and effect exceeds the present normal range of the plan (743);

(4) notifying the inhabitant the abnormal situations through mobile communication device, E-mail or telephone etc. communication media (744);

(5) determining whether the management period is ended (745), wherein if the management period is not ended, go to step (1); if not, the next step is executed;

(6) transmitting a execution record file to the corresponding community management server to provide the inhabitant an complete execution report including all detail executing records and results (746); and

(7) cyclically going back to the first step: measuring and estimating basic health conditions procedure (747).

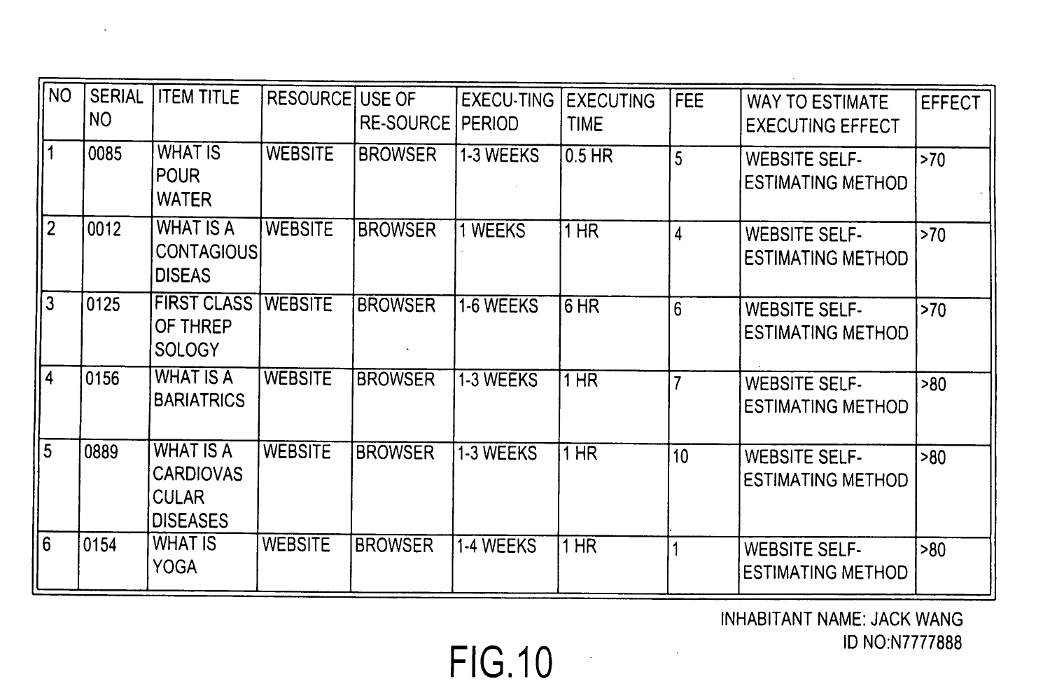

Since the exercise equipment(21), the biotech measuring equipment(22), the healthcare equipment(23), the multimedia playing equipment(24) and the printing equipment(25) link to the center server (10) through the community management server (20), the center server sets the procedures for different equipment according to the personal health management plan and also reads the executed records of the equipment or transmits an proper exam to the inhabitant executing his or her own health management plan. With further reference to FIG. 10, a knowledge suggestion list of the personal health management plan includes different knowledge suggestion items, resources of obtaining the contents of the knowledge suggestion items, executing period and time of each suggestion item, way of estimating executing effect and effect for each suggestion item. Since the center server has to ensure the effect of executing knowledge suggestion list, the center server will transmits an exam for each suggestion item to the inhabitant after the inhabitant studied the content of the suggestion item. Thus, the center server successfully manages the inhabitant's executing procedure and provides the inhabitant a reliable management report of inhabitant's health. If the inhabitant repeats the five steps, the number of abnormal items of the examination should be decreasing gradually as shown in FIG. 7.

During executing the personal health management plan, the inhabitant would like to have necessary goods, materials or services related to healthcare. Therefore, the center server could links to the goods/materials/services supplier to order and then the goods/material/service trading server notifies the delivery company through the delivery trading server to delivery objects. In addition, the delivery company could be a health center, so the health center sends a vehicle to inhabitant's home to carry the inhabitant to execute body suggestion items in the health center, or sends a hygienist to tie inhabitant's home to provide a specific service for executing the environment suggestion items.

Based on the forgoing description, the center server links to many community management servers, a hospital server, a professional consulting server and a goods/materials/services supplier trading server to provide the inhabitant information related health and hygiene cares and manage the inhabitant to execute the personal health management plan. Further, the center server links to the internet bank trading server and the medical insurance service server to provide the inhabitant to estimate risk and to adjust his or her insurance content. The present invention has following advantages:

1. Since the center server could link to many associated community management server, the present invention is able to serves many community inhabitants. Therefore, the building system cost is not too expensive and the consuming risk and long-term consuming cost are decreased.

2. Since the present invention provides each inhabitant a personal health management plan according to the conditions of his or her life story, environment and body and mind, the unnecessary management cost, risk, and responsibility is eliminated.

3. Since the present invention also provides knowledge related of the health and hygiene cares, the inhabitant also studies the content of the knowledge. Therefore, the personal knowledge level of the health and hygiene cares and the qualities of health management and risk management are increased and the preventive medicine will be universal gradually.

4. Executing a personal health management will be universal gradually.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A health management trading method, comprising steps of:
(a) estimating basic health conditions of each inhabitant;
(b) selecting proper items of a health examination to examine the actual inhabitant's body and mind;
(c) providing professional consulting and suggesting for the proper solution items of a health management plan according to the estimating results in steps (a) and (b);
(d) selecting and registering a formal personal health management plan according to the professional consulting and suggesting results; and
(e) executing formal personal health management plan and reporting abnormal situation and executing effects, wherein the executing formal personal health management plan including setting and tracking the executing procedure to find out the abnormal situation and the executing effects.

2. The cyclic service method as claimed in claim 1, wherein in the step (a), the estimating basic health conditions comprises community environment quality, health knowledge level and body and mind health.

3. The cyclic service method as claimed in claim 2, wherein in step (b), the suggesting result includes environment suggestions, knowledge suggestions, exercise suggestions, drink and food suggestions, risk management suggestions and healthcare suggestion.

4. The cyclic service method as claimed in claim 3, wherein the step (a) comprises acts of:
(1) measuring the community environment quality;
(2) estimating health conditions of life history, knowledge, and body and mind of the inhabitant;
(3) storing measuring values and estimating results in a personal basic health condition estimation database;
(4) executing an intersected analysis operation to generate a personal health examination suggestion list according to information of the personal basic health condition estimation database;
(5) transmitting a personal health examination suggestion list having examining items to the corresponding inhabitant; and
(6) end the step (a).

5. The cyclic service method as claimed in claim 4, wherein the step (b) comprises acts of:
(a) being in consultation with a proper professional consultant to know content of the examining items on the personal health examination suggestion list;
(b) selecting proper examining items on the personal health examination suggestion list;
(c) registering a formal health examination plan generated by selected examining items;
(d) paying the formal health examination fee;
(e) notifying the inhabitant to complete the formal health examination;
(f) obtaining the inhabitant's formal health examination results;
(g) transmitting the inhabitant's formal health examination results to the inhabitant; and
(h) end the step (b).

6. The cyclic service method as claimed in claim 5, wherein the step (c) comprises acts of:
(1) checking the examining result for each abnormal item, wherein the examining result shows medical title, normal range, examined value and related risk;
(2) being in consultation with consultants through the professional consulting server to know information related to all abnormal examining items or to have proper medical treatments, the inhabitant should be necessarily done;
(3) providing the inhabitant a personal health management plan suggestion including environment solution suggestion, knowledge solution suggestion, exercise solution suggestion, drink and food solution suggestion, risk solution suggestion and health care solution suggestion according to the personal basic health condition estimation result and the formal personal health examination result;
(4) transmitting the personal health management plan suggestion to the corresponding community management server and further providing the inhabitant a report of the personal health management plan suggestion; and
(5) end the step (c).

7. The cyclic service method as claimed in claim 6, wherein the step (d) comprises acts of:
(1) being in consultation with professional consultants to know each suggestion item;
(2) selecting proper suggestion items of a personal health management plan suggestion list to build a formal personal health management plan;
(3) registering the formal personal health management plan;
(4) paying the fee of the formal personal health management plan;
(5) notifying the corresponding inhabitant to execute the formal personal health management plan; and
(6) end step (d).

8. The cyclic service method as claimed in claim 7, wherein the step (e) comprises acts of:
(1) continually setting corresponding equipment's condition and reading executed records during executing the personal health management plan;
(2) occasionally being in consultation with the professional consultants to further fine latent diseases easily according to problems during executing the personal health management plan;
(3) determining whether an executing schedule and effect exceeds the present normal range of the plan;
(4) notifying the inhabitant the abnormal situations through communication media;
(5) determining whether the health plan is ended, wherein if the management period is not ended, go to step (1), but if not, the next step is executed;
(6) transmitting a execution record file to the corresponding community management server to provide the inhabitant an complete execution report including all detail executing records and results; and
(7) cyclically going back to the first step: measuring and estimating basic health conditions procedure.

9. A health management cyclic service system, comprising:
a center server building a health management procedure;
a community management server linking to the center server to serve community inhabitants more effectively and more conveniently;
a professional consulting server linking to the center server and at least one community management server, wherein the professional consulting server provides medical information and health management plan suggestion;
an internet bank trading server linking to the center server to execute a dollar trading procedure;
a hospital server linking to the center server to provide the center server health examination reports;
a supplier trading server linking to the center server to supply customers all needed goods, materials and services; and
a multimedia server linking to the center server to provide educational contents related to medicine, health management and healthcare.

10. The cyclic service system as claimed in claim 9, wherein the center server further builds a community association customer database, a hospital and supplier database, a medical disease database, a preventive medicine database, a knowledge solution database, an environment solution database, a drink and food solution database, an exercise solution database, a risk management solution database, a healthcare solution database, a result of health examination database, a consultant database, a health examination basic database, an accounting ledger database and a basic health condition measuring/estimating result database.

11. The cyclic service system as claimed in claim 10, wherein the center server further comprises:
a website management and basic health condition estimating processing module having a website basic database, an estimation content database and a product and service supplier database;
a website trading and professional consulting fee processing module having a web trading record database and a consulting fee record database;
a cyclic health management processing module having a cyclic health management basic database and a cyclic health management plan changed record database; and
an effect tracking and reporting processing module having a formal health management plan database and an executing progress record database.

12. The cyclic service system as claimed in claim 10, wherein the health management cyclic service method of the center server comprises steps of:
(a) estimating a community inhabitants' basic health conditions of environment, knowledge, body and mind;
(b) scheming a personal health examination suggestion list according to the estimating results and then providing the personal health examination suggestion list to corresponding community inhabitant to select the proper examining items of the health examination on the suggestion list;
(c) determining whether the community inhabitant selects and examines any examining item on personal health examination suggestion list, wherein if the community inhabitant does not select any examining item and then the health management procedure is end, but if the community inhabitant selects at least one examining item on the health examination suggestion list, the next step is executed;
(d) providing a health examination results to the corresponding community inhabitant;
(e) providing a set of different kind professional health management solution suggestions to the corresponding community inhabitant according to examining results, wherein each kind of the professional solution suggestion contains at least one solution to be executed in required condition;
(f) determining whether the community inhabitant selects and registers any solution from the professional health management solution suggestions, wherein if the community inhabitant does not register any solution and then the health management procedure is end, but if the community inhabitant registers at least one solution, a formal personal health management plan is schemed according to the selected solutions and then the nest step is executed;
(g) setting and tracking an executing procedure of the formal personal health management plan and notifying the corresponding community inhabitant any abnormal situation; and
(h) generating a final execution report when the formal personal health management plan is ended.

13. The cyclic service system as claimed in claim 12, wherein in the step (c) if the community inhabitant wants to attend at least one health examination article on personal health examination list, the community inhabitant has to pay examination fee to determine attend the health examination article.

14. The cyclic service system as claimed in claim 9, wherein the center server further links to a goods/materials/services supplier trading server and provides a purchase service.

15. The cyclic service system as claimed in claim 9, wherein the effect tracking and reporting processing module of the center server further links to a multimedia server storing health and hygiene education data, wherein the multimedia server sends a suitable health and hygiene education data to corresponding community's multimedia playing equipment to display.

16. The cyclic service system as claimed in claim 9, wherein each community management server has an effect tracking and reporting processing module having a formal health management plan database and executing progress record database.

17. The cyclic service system as claimed in claim 9, wherein the community management server further links to at least one specific health management equipment comprising:
a host controller having a central processor with a memory unit, a communication interface controller, an audio and video output processor, a specific function module, an user input device and a power unit, wherein the central processor links to the communication interface controller, the audio and video output processor, the user input device and the memory unit;
an identity recognizing device connected to the host controller; and
an audio and video output device links to the audio and video output processor to present audio and video signals.

18. The cyclic service system as claimed in claim 17, wherein the at least one specific health management equipment is an exercise equipment, a biotech measuring equipment, a healthcare equipment, a multimedia playing equipment or a printing equipment.
